SEQUENCE LISTING (4 sequences)

SEQ ID NO: 1 - protein, 24 aa, Artificial Sequence
  Synthetic fusion protein
SLLTEVETPIRNEWGSRSNDSSDP

SEQ ID NO: 2 - protein, 27 aa, Artificial Sequence
  Synthetic fusion protein
ELEPPFGDSYIVVGRGEQQINHHWHKS

SEQ ID NO: 3 - protein, 27 aa, Artificial Sequence
  Synthetic fusion protein
EMEPPFGDSYIVVGMGDKQINHHWHKA

SEQ ID NO: 4 - protein, 27 aa, Artificial Sequence
  Synthetic fusion protein
EAEPPFGDSYIIIGVEPQQLKLNWFKK

(19) United States
(12) Patent Application Publication
(10) Pub. No.: US 2009/0136537 A1
(43) Pub. Date: May 28, 2009

(54) Lipopeptide compositions and methods of use therof

(76) Inventors: Robert Evans, Souderton, PA (US); David Jackson, New York, NY (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/214,421

(22) Filed: Jun. 18, 2008

Related U.S. Application Data
(63) Continuation of application No. PCT/US2006/047961, filed on Dec. 15, 2006.
(60) Provisional application No. 60/752,932, filed on Dec. 21, 2005.

(51) Int. Cl. A61K 39/00

(52) U.S. Cl. 424/192.1; 424/184.1

(57) ABSTRACT

Compositions comprise lipopeptides, antigens and emulsifying agents. The compositions are used to stimulate an immune response in a subject.

### RELATED APPLICATION(S)

This application is a continuation of International Application No. PCT/US2006/047961, which designated the United States and was filed on Dec. 15, 2006, published in English, which claims the benefit of U.S. Provisional Application No. 60/752,932, filed on Dec. 21, 2005. The entire teachings of the above applications are incorporated herein by reference.

### BACKGROUND OF THE INVENTION

Compositions comprising hydrophobic molecules, such as lipopeptides, can have limited aqueous solubility, which can affect the preparation of sterile forms of the compositions for use in, for example, the treatment of a subject. Currently available compositions that include hydrophobic molecules can be prepared by employing, for example, organic solvents and acetate acid to assist in solubilization of the hydrophobic molecules in such compositions. However, such preparations may require further processing, which can result in dilution and alterations in the chemical nature of the compositions and, thus, inadequate compositions for use in the treatment of subjects. Thus, there is a need to develop new compositions that contain hydrophobic molecules for use in treating subjects.

### SUMMARY OF THE INVENTION

The present invention relates to compositions comprising lipopeptides and antigens. The compositions can be employed in methods to stimulate an immune response in a subject.

In one embodiment, the invention is a composition comprising a lipopeptide, an antigen and an emulsifying agent, wherein the lipopeptide includes at least one member selected from the group consisting of Pam3Cys and Pam2Cys.

In another embodiment, the invention is a composition comprising a lipopeptide, an antigen, a polyoxyethylene sorbitan monoleate, a cyclodextrin, a docusate salt and a surfactant.

In an additional embodiment, the invention is a composition comprising a lipopeptide, an antigen and a polyoxyethylene sorbitan monoleate, wherein the lipopeptide includes at least one member selected from the group consisting of Pam3Cys and Pam2Cys.

In still another embodiment, the invention is a composition comprising a lipopeptide, an antigen and a docusate salt, wherein the lipopeptide includes at least one member selected from the group consisting of Pam3Cys and Pam2Cys.

In a further embodiment, the invention is a composition comprising a lipopeptide, an antigen and a surfactant, wherein the lipopeptide includes at least one member selected from the group consisting of Pam3Cys and Pam2Cys.

In another embodiment, the invention is a composition comprising a lipopeptide, an antigen and a cyclodextrin, wherein the lipopeptide includes at least one member selected from the group consisting of Pam3Cys and Pam2Cys.

In still another embodiment, the invention is a method of stimulating an immune response in a subject, comprising the step of administering to the subject a composition that includes a lipopeptide, an antigen and an emulsifying agent, wherein the lipopeptide includes at least one member selected from the group consisting of Pam3Cys and Pam2Cys.

In yet another embodiment, the invention is a method of stimulating an immune response in a subject, comprising the step of administering to the subject a composition that includes a lipopeptide, an antigen, a polyoxyethylene sorbitan monoleate, a cylcodextrin, a docusate salt and a surfactant.

An additional embodiment of the invention is a method of stimulating an immune response in a subject, comprising the step of administering to the subject a composition that includes a lipopeptide, an antigen and a polyoxyethylene sorbitan monoleate, wherein the lipopeptide includes at least one member selected from the group consisting of Pam3Cys and Pam2Cys.

Another embodiment of the invention is a method of stimulating an immune response in a subject, comprising the step of administering to the subject a composition that includes a lipopeptide, an antigen and a docusate salt, wherein the lipopeptide includes at least one member selected from the group consisting of Pam3Cys and Pam2Cys.

A further embodiment of the invention is a method of stimulating an immune response in a subject, comprising the step of administering to the subject a composition that includes a lipopeptide, an antigen and a surfactant, wherein the lipopeptide includes at least one member selected from the group consisting of Pam3Cys and Pam2Cys.

In yet another embodiment, the invention is a method of stimulating an immune response in a subject, comprising the step of administering to the subject a composition that includes a lipopeptide, an antigen and a cyclodextrin, wherein the lipopeptide includes at least one member selected from the group consisting of Pam3Cys and Pam2Cys.

The compositions of the invention can have increased aqueous solubility and, thus, can be sterile filtered for use in treating a subject. The compositions of the invention can be employed to stimulate an immune response in a subject.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 depicts a Pam3Cys.M2e fusion protein. The amino acid sequence (SEQ ID NO: 1) of M2e is shown in bold type.

FIG. 2 depicts the effect of the concentration of docusate sodium (DS) on M2e ELISA response to Pam3Cys.M2e.

FIG. 3 depicts a Pam3Cys.M2e efficacy study survival.

### DETAILED DESCRIPTION OF THE INVENTION

A description of example embodiments of the invention follows.

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

In one embodiment, the invention is a composition comprising a lipopeptide, an antigen and an emulsifying agent, wherein the lipopeptide includes at least one member selected from the group consisting of Pam3Cys and Pam2Cys. Pam3Cys is also referred to herein as “[Palmitoyl]-Cys((RS)-2,3-di(palmitoyloxy)-propyl cysteine” and “P2.” Pam2Cys is also referred to as “S-[2,3-bis(palmitoyloxy) propyl] cysteine.” Pam2Cys and Pam3Cys are Toll-like receptor 2 (TLR2) agonists.

“Lipoprotein,” as used herein, refers to a molecule having at least one amino acid that includes a lipid component attached to or associated with the amino acid. The attachment to or association with the amino acid can be a covalent or a noncovalent attachment. In a particular embodiment, the lipopeptide is a Pam3Cys and a Pam2Cys.

The compositions can include, for example, two, three, four, five, six or more lipopetides (e.g., Pam2Cys, Pam3Cys) and two, three, four, five, six or more antigens (e.g., integral membrane proteins of an influenza viral protein, a flavirus). When two or more lipopeptides and/or two or more antigens comprise the compositions of the invention, they are also referred to as “multimers.” For example, a multimer of the amino-terminus of an M2 protein can be four, 24-amino acid sequences (total of 96 amino acids), which is referred to herein as 4×M2 or 4×M2e (“M2e” refers to the 24 amino acid amino-terminus of the M2 protein or its ectodomain).

The lipopeptide and the antigen can be components of a fusion protein.

“Fusion protein,” as used herein, refers to a protein generated from at least two similar or distinct components (e.g., lipopeptides, such as Pam2Cys, Pam3Cys; at least a portion of an antigen, such as an integral membrane protein of an influenza viral protein (M2 protein) or a flavivirus) that are linked covalently or noncovalently. The components of the fusion protein can be made, for example, synthetically (e.g., Pam3Cys, Pam2Cys) or by recombinant nucleic acid techniques (e.g., transfection of a host cell with a nucleic acid sequence encoding a component of the fusion protein, such as at least a portion of a peptide comprising the lipopeptide, or at least a portion of an antigen, such as an integral membrane protein of an influenza viral protein or a flavivirus). One component of the fusion protein (e.g., Pam2Cys, Pam3Cys, an antigen) can be linked to another component of the fusion protein (e.g., Pam2Cys, Pam3Cys, an antigen) using chemical conjugation techniques, including peptide conjugation, or using molecular biological techniques, including recombinant technology, such as the generation of a fusion protein construct.

“Antigen,” as used herein, refers to any molecule (e.g., a protein, peptide, polypeptide, carbohydrate, glycoprotein) that generates an immune response in a subject (e.g., mice, rat, rabbit, ferret, monkey, human).

The emulsifying agent in the compositions of the invention include at least one member selected from the group consisting of a polysorbate, a cyclodextrin, a docusate salt and a surfactant.

Exemplary polysorbates for use in the compositions of the invention include polysorbate-20 (also referred to as “polyoxyethylene sorbitan monolaurate”), polysorbate-40 (also referred to as “polyoxyethylene sorbitan monopalmitate”); polysorbate-60 (also referred to as “polyoxyethylene sorbitan monosterate”); and polysorbate-80 (also referred to as “polyoxyethylene sorbitan monooleate”).

The salt of a docusate can be a sodium salt (Bis(2-ethylhexyl)Sulfosuccinate), a lithium salt or a potassium salt.

Exemplary cyclodextrins for use in the invention include sulfobutylether-beta-cyclodextrin and hydroxypropyl-beta-cyclodextrin.

The surfactant can be a negatively charged surfactant (also referred to herein as an “anionic surfactant”), a positively charged surfactant (also referred to herein as a “cationic surfactant”) and a zwitterionic surfactant. Negatively charged surfactants in the compositions of the invention can include, for example, Dilauroylphosphoglycerol (1,2-Dilauroyl-sn-Glycero-3-[Phospho-rac-(1-glycerol)]; phosphatidic acid; saturated fatty acids, such as lauric acid, myristic acid, palmitic acid and stearic acid; unsaturated fatty acids, such as palmitoleic acid, oleic acid, linoleic acid and linolenic acid; deoxycholic acid; cholic acid; caprylic acid; glycocholic acid; glycodeoxycholic acid; lauroylsarcosine; and n-dodecyl sulfate.

Exemplary positively charged surfactants for use in the compositions of the invention can include benzalkonium chloride (alkylbenzyldimethylammonium chloride); cetylpyridinium chloride; and cetyltrimethylammonium chloride (hexadecyltrimethylammonium chloride).

Exemplary zwitterionic surfactants for use in the compositions of the invention can include phosphatidylcholine (1,2-Diacyl-sn-glycero-3-phophocholine); CHAPS (3-[(3-Cholamidopropyl)dimethylammonio]-1-propansulfonate); BigCHAP (N,N-Bis[3-(D-gluconamido)propyl]cholamide); and CHAPSO (3-[(3-Cholamidopropyl)dimethylammonio]-2-hydro-xy-1-propanesulfonate).

In a further embodiment, the invention is a composition comprising a lipopeptide (e.g., Pam2Cys, Pam3Cys), an antigen, a polysorbate (e.g., polyoxyethylene sorbitan monoleate), a cyclodextrin, a docusate salt and a surfactant.

In an additional embodiment, the invention is a composition comprising a lipopeptide, an antigen and a polysorbate, wherein the lipopeptide includes at least one member selected from the group consisting of Pam3Cys and Pam2Cys.

In yet another embodiment, the invention is a composition comprising a lipopeptide, an antigen and a docusate salt, wherein the lipopeptide includes at least one member selected from the group consisting of Pam3Cys and Pam2Cys.

In still another embodiment, the invention is a composition comprising a lipopeptide, an antigen and a surfactant, wherein the lipopeptide includes at least one member selected from the group consisting of Pam3Cys and Pam2Cys.

In another embodiment, the invention is a composition comprising a lipopeptide, an antigen and a cyclodextrin, wherein the lipopeptide includes at least one member selected from the group consisting of Pam3Cys and Pam2Cys.

An additional embodiment of the invention is a method of stimulating an immune response in a subject, comprising the step of administering to the subject a composition that includes a lipopeptide, an antigen and an emulsifying agent, wherein the lipopeptide includes at least one member selected from the group consisting of Pam3Cys and Pam2Cys.

“Stimulating an immune response,” as used herein, refers to the generation of antibodies to at least a portion of an antigen (e.g., an integral membrane, such as M2, HA, NA of influenza A, B and/or C, flavivirus, such as the West Nile virus). Stimulating an immune response in a subject can include the production of humoral and/or cellular immune responses that are reactive against the influenza viral protein. In stimulating an immune response in the subject, the subject may be protected from infection by the antigen that may diminish or be halted as a consequence of stimulating an immune response in the subject.

In another embodiment, the invention is a method of stimulating an immune response in a subject, comprising the step of administering to the subject a composition that includes a lipopeptide, an antigen, a polysorbate (i.e., polyoxyethylene sorbitan monoleate), a cylcodextrin, a docusate salt and a surfactant.

In a further embodiment, the invention is a method of stimulating an immune response in a subject, comprising the step of administering to the subject a composition that includes a lipopeptide, an antigen and a polysorbate, wherein the lipopeptide includes at least one member selected from the group consisting of Pam3Cys and Pam2Cys.

In yet another embodiment, the invention is a method of stimulating an immune response in a subject, comprising the step of administering to the subject a composition that includes a lipopeptide, an antigen and a docusate salt, wherein the lipopeptide includes at least one member selected from the group consisting of Pam3Cys and Pam2Cys.

In still another embodiment, the invention is a method of stimulating an immune response in a subject, comprising the step of administering to the subject a composition that includes a lipopeptide, an antigen and a surfactant, wherein the lipopeptide includes at least one member selected from the group consisting of Pam3Cys and Pam2Cys.

Another embodiment of the invention is a method of stimulating an immune response in a subject, comprising the step of administering to the subject a composition that includes a lipopeptide, an antigen and a cyclodextrin, wherein the lipopeptide includes at least one member selected from the group consisting of Pam3Cys and Pam2Cys.

The methods of the present invention can be accomplished by the administration of the compositions of the invention by enteral or parenteral means. Specifically, the route of administration is by oral ingestion (e.g., drink, tablet, capsule form) or intramuscular injection of the compositions of the invention. Other routes of administration as also encompassed by the present invention including intravenous, intradermal, intraarterial, intraperitoneal, or subcutaneous routes, and nasal administration. Suppositories or transdermal patches can also be employed.

The compositions of the invention can be administered alone or can be coadministered to the subject. Coadminstration is meant to include simultaneous or sequential administration of one or more of the compositions of the invention individually or in combination. Where one or more compositions is administered, the mode of administration can be conducted sufficiently close in time to each other (for example, administration of the composition close in time to administration of another composition) so that the effects on stimulating an immune response in a subject are maximal. It is also envisioned that multiple routes of administration (e.g., intramuscular, oral, transdermal) can be used to administer the compositions of the invention.

The compositions of the invention can be administered alone or as admixtures with conventional excipients, for example, pharmaceutically, or physiologically, acceptable organic, or inorganic carrier substances suitable for enteral or parenteral application which do not deleteriously react with the extract. Suitable pharmaceutically acceptable carriers include water, salt solutions (such as Ringer's solution), alcohols, oils, gelatins and carbohydrates such as lactose, amylose or starch, fatty acid esters, hydroxymethycellulose, and polyvinyl pyrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like which do not deleteriously react with the compositions of the invention. The preparations can also be combined, when desired, with other active substances to reduce metabolic degradation. The compositions of the invention can be administered by oral administration, such as a drink, intramuscular or intraperitoneal injection. The compositions alone, or when combined with an admixture, can be administered in a single or in more than one dose over a period of time to confer the desired effect (e.g., alleviate or prevent viral infection, to alleviate symptoms of viral infection).

When parenteral application is needed or desired, particularly suitable admixtures for the compositions are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-block polymers, and the like. Ampules are convenient unit dosages. The compositions can also be incorporated into liposomes or administered via transdermal pumps or patches. Pharmaceutical admixtures suitable for use in the present invention are well-known to those of skill in the art and are described, for example, in Pharmaceutical Sciences (17th Ed., Mack Pub. Co., Easton, Pa.) and WO 96/05309 the teachings of which are hereby incorporated by reference.

The dosage and frequency (single or multiple doses) administered to a subject can vary depending upon a variety of factors, including prior exposure to a viral antigen, the duration of viral infection, prior treatment of the viral infection, the route of administration of the composition; size, age, sex, health, body weight, body mass index, and diet of the subject; nature and extent of symptoms being treated (e.g., influenza infection) kind of concurrent treatment (e.g., drugs), complications from the condition being treated or other health-related problems. Other therapeutic regimens or agents can be used in conjunction with the methods and compositions of the present invention. For example, the administration of the compositions can be accompanied by other viral therapeutics or use of agents to treat the symptoms of the condition or disease being treated (e.g., influenza infection treatment with nasal sprays and drugs, such as amantadine, rimantadine, zanamivir and oseltamivir). Adjustment and manipulation of established dosages (e.g., frequency and duration) are well within the ability of those skilled in the art.

The present invention is further illustrated by the following examples, which are not intended to be limiting in any way.

### EXEMPLIFICATION

### Example 1

### Preparation of Compositions

### Materials and Methods:

Materials Hydroxypropyl-B-cyclodextrin (HPBCD) was purchased from Research Diagnostics (Flanders, N.J., catalog #RDI-410200). Polysorbate-80 was purchased from Croda Inc. (Edison, N.J., product name CRILLET 4 HP). Dilauroylphosphoglycerol (1,2-Dilauroyl-sn-Glycero-3-[Phospho-rac-(1-glycerol)] (DLPG) was purchased from Avanti Polar Lipids, Inc. (Alabaster, Ala.). PBS (phosphate buffered saline) was purchased from Fisher Scientific (Morris Plains, N.J.). The 14C2 and biotinylated 14C2 monoclonal antibodies were purchased from Affinity BioReagents (Golden, Colo.). Assay diluent (AD), Avidin-HRP and Ultra TMB substrate were purchased from BD BioScience (San Jose, Calif.).

Synthesis and purification of Pam3Cys Peptides were purchased from Genemed Synthesis Inc. (San Francisco, Calif.) or Bachem AG (Bubendorf, Switzerland). All peptides were synthesized using solid phase Fmoc synthesis methodologies and purified by reverse phase HPLC. Mass spectroscopy analysis was used to verify the molecular weight of the final product.

Preparation of formulation buffers All the buffers except PBS were prepared by dissolving the formulation excipients in deionized water and adjusting the pH to about 7.2 at about 25° C. with 1 M NaOH. After adjusting to the final volume with deioinized water the buffers were sterilized by filtering through a sterile 0.22 ÿm pore size membrane. Filter sterilized buffers were stored at about 2-8° C. The components of the formulation buffers and designations of the buffers are listed in the Table 1, infra.

Compositions Unless stated otherwise lipopeptides (also referred to herein as “lipopeptides”) and antigens were formulated in the various formulation buffers by resuspending dry powder peptides in sterile formulation buffer pre-warmed to about 37° C. and vigorously mixing by high speed vortexing for 1-3 minutes. All samples were prepared at a peptide concentration of 1 mg/mL. Typically, samples were then incubated at about 37° C. for an additional 1-3 hours followed by an additional 1-3 minutes of vortexing. Samples were then filtered through a 33 mm diameter syringe filter equipped with a PVDF membrane having a 0.22 ÿm pore size.

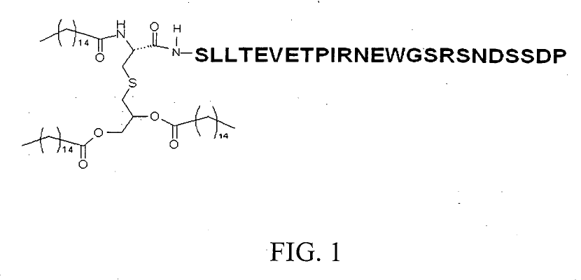

UV absorbance spectroscopy UV absorbance measurements were performed to determine the peptide concentration and the A280/A350 ratio. For Pam3Cys.M2e (FIG. 1) the peptide concentration was determined using the following equation.

Pam3Cys.M2e concentration (mg/mL)=(A280-A350)1.59

Particle size measurements Particle size distributions were determined by dynamic light scattering using a Malvern Zetasizer Nano ZS (Malvern, Pa.). The refractive index of the peptides was set to 1.45 while the refractive index for the F111 dispersant was set to 1.27. The viscosity of the F111 dispersant was set to 1.1463 and all measurements were performed at 25° C. in a low volume glass cuvette. For each particle size determination a total of 15 measurements was performed with each measurement being 10 seconds in duration.

M2e ELISA The M2e ELISA was used to assess the antigenicity of Pam3Cys.M2e compositions. The assay is a sandwich ELISA using the mAb 14C2 to capture the Pam3Cys.M2e particles on the ELISA plate and a biotinylated version of the same mAb (14C2b) for detection. The ELISA plate was first coated with 100 ÿL/well of 14C2 mAb in PBS at a concentration of 2 ÿg/mL. The 14C2 mAb was allowed to bind to the plate overnight at 4° C. After removing the 14C2 mAb solution from the plates the wells were blocked for 90 minutes at room temperature using 300 ÿL of assay diluent in each well. The plates were then washed two times with PBS and blotted dry. Pam3Cys.M2e was prepared in F111 and F113 containing various concentrations of docusate sodium to a final peptide concentration of 1 mg/mL. Pam3Cys.M2e samples were diluted with 30% assay diluent (70% PBS, 30% assay diluent) to 1 ÿg/mL and placed in one column of the ELISA plate (seven replicates) and incubated at room temperature for one hour. The plates were then washed three times with PBS and blotted dry. A 1 ÿg/mL concentration of biotinylated detection mAb (14C2b) in 30% assay diluent was then added to each well (100 ÿL per well) and incubated for one hour at room temperature. The plates were then washed three times with PBS and blotted dry. Then, 100 ÿL of Avidin-HRP (Avidin-horse radish peroxidase) diluted 1:2000 in 30% assay diluent was added to each well and the plates were incubated for 30-45 minutes at room temperature. The plates were again washed three times with PBS and blotted dry. To develop the ELISA plates 100 ÿL of Ultra TMB substrate was added to each well. Color development was allowed to proceed until the blue color in the wells reached an optical density near 2.0 (approximately 5 minutes). Color development was stopped by adding 100 ÿL of 1 M sulfuric acid to each well. The absorbance (Abs) of the plate was then read at 450 nm. The Abs for each formulation was determined by averaging the absorbance reading for the seven replicates for each sample.

BALB/c mouse efficacy study Female BALB/c mice (10 animals per group, 5-6 weeks old) were obtained and allowed to acclimate for one week. Pam3Cys.M2e formulated in F111, F119 and F120 at 0.3 mg/mL were administered by s.c. injection (30 ÿg dose). The control group was immunized with PBS. The convalescent group was a group which had successfully cleared an earlier non-lethal infection with PR/8. Mice were immunized on days 0 and 14. On day 21, sera were harvested by retro-orbital puncture. Mice were challenged by intranasal administration of 1×LD90 of the well characterized mouse adapted Influenza A strain, A/Puerto Rico/8/34 (H1N1). Mice were monitored daily for 14 days for survival. Mice that lose 30% of their initial body weight are humanely sacrificed, and the day of sacrifice is recorded as the day of death.

TABLE 1
FORMULATION DESCRIPTIONS

| Formu- | |
|---|---|
| lation | Components of Formulation |
| F101 | PBS (phosphate buffered saline; 2.7 mM KCl, 1.5 mM |
| | KH₂PO₄, 137 mM NaCl, 8.1 mM Na₂PO₄, pH 7.4) |
| F105 | 10 mM Tris, 10 mM histidine, 5% sucrose, 75 mM NaCl, |
| | 0.02% polysorbate-80 (PS-80), 0.1 mM EDTA, 0.5% (v/v) |
| | ethanol, pH 7.2 |
| F106 | 10 mM Tris, 10 mM histidine, 5% sucrose, 75 mM NaCl, 0.1 |
| | mM EDTA, 0.5% (v/v) ethanol, pH 7.2 |
| F107 | 10 mM Tris, 10 mM histidine, 5% sucrose, 75 mM NaCl, pH |
| | 7.2 |
| F108a | 2.7 mM KCl, 1.5 mM KH₂PO₄, 137 mM NaCl, 8.1 mM |
| | Na₂PO₄, 0.02% PS-80, pH 7.4 |
| F108 | 2.7 mM KCl, 1.5 mM KH₂PO₄, 137 mM NaCl, 8.1 mM |
| | Na₂PO₄, 0.5% PS-80, pH 7.4 |
| F109 | 2.7 mM KCl, 1.5 mM KH₂PO₄, 137 mM NaCl, 8.1 mM |
| | Na₂PO₄, 20 mg/mL hydroxypropyl-ÿ-cyclodextrin (HPBCD), |
| | pH 7.4 |
| F110 | 10 mM Tris, 10 mM histidine, 5% sucrose, 75 mM NaCl, 0.1 |
| | mM EDTA, 0.5% (v/v) ethanol, 20 mg/mL HPBCD, pH 7.2 |
| F111 | 10 mM Tris, 10 mM histidine, 5% sucrose, 75 mM NaCl, |
| | 0.02% polysorbate-80 (PS-80), 0.1 mM EDTA, 0.5% (v/v) |
| | ethanol, 20 mg/mL HPBCD, pH 7.2 |
| F113 | 10 mM Tris, 10 mM histidine, 10% sucrose, 0.02% |
| | polysorbate-80 (PS-80), 0.1 mM EDTA, 0.5% (v/v) ethanol, |
| | pH 7.2 |
| F119 | 10 mM Tris, 10 mM histidine, 5% sucrose, 75 mM NaCl, |
| | 0.02% polysorbate-80 (PS-80), 0.1 mM EDTA, 0.5% (v/v) |
| | ethanol, 20 mg/mL HPBCD, 0.1% docusate sodium, pH 7.2 |
| F120 | 10 mM Tris, 10 mM histidine, 10% sucrose, 0.02% |
| | polysorbate-80 (PS-80), 0.1 mM EDTA, 0.5% (v/v) ethanol, |
| | 0.075% docusate sodium, pH 7.2 |
| F123 | 10 mM Tris, 10 mM histidine, 10% sucrose, 0.02% |
| | polysorbate-80 (PS-80), 0.1 mM EDTA, 0.5% (v/v) ethanol, |
| | 0.1% DLPG, pH 7.2 |

### Example 2

### Polysorbate-80 and Hydroxypropyl-ÿ-Cyclodextrin Enhance the Solubility of Compositions Comprising a Lipopeptide and an Antigen

To determine the effect of polysorbate-80 (PS-80) and hydroxypropyl-ÿ-cyclodextrin (HPBCD) on the solubility of a composition comprising a lipopeptide (Pam3Cys) and an antigen (M2e) (the Pam3Cys.M2e fusion protein), four formulations (the components of which are shown in the Table 1, supra) were prepared, each containing 1 mg/mL Pam3Cys.M2e. Pam3Cys.M2e was dispensed into each formulation at room temperature and mixed by vortexing. After mixing, the UV absorbance spectrum of Pam3Cys.M2e was determined in each formulation, compared to the same formulation without Pam3Cys.M2e as the blank. The UV absorbance of Pam3Cys.M2e at 280 nm is due entirely to the presence of tryptophan in the peptide sequence. However, because tryptophan does not absorb light at 350 nm any absorbance at this wavelength is due to light scattering caused by particles of Pam3Cys.M2e that are not in solution. Therefore, a higher A280/A350 absorbance ratio is indicative of higher solubility.

The A280/A350 ratios of the test formulations are shown in Table 2 below. The results indicate that the A280/A350 ratio for Pam3Cys.M2e in F108a, F108 and F109 (see Table 1, supra) was significantly higher than the A280/A350 ratio for Pam3Cys.M2e in PBS (3.9). The higher A280/A350 ratio for the formulations containing PS-80 or HPBCD indicate that Pam3Cys.M2e is more soluble in formulations F108a, F108 and F109 than it is in F101.

TABLE 2
Effect of PS-80 and HPBCD on Pam3Cys.M2e solubility.

| Formulation | A280/A350 | Filtration Recovery |
|---|---|---|
| F101 | 3.9 | 58% |
| F108a | 5.6 | 78% |
| F108 | 8.9 | 85% |
| F109 | 8.6 | 86% |

To determine the effect of PS-80 and HPBCD on the recovery of Pam3Cys.M2e through a filtration process, about 1 mL of each test formulation was filtered through a 33 mm diameter syringe filter with a 0.22 ÿm pore size PVDF membrane. The concentration of Pam3Cys.M2e was determined before and after filtration after correction for light scattering. The light scattering correction was performed by subtracting the absorbance at 350 nm from the absorbance at 280 nm. The results (Table 2) clearly show that the filtration recovery of Pam3Cys.M2e was significantly higher in the formulations containing either PS-80 or HPBCD, compared to the PBS control. Taken together, these results indicate that PS-80 and HPBCD enhance the solubility of Pam3Cys.M2e in PBS.

### Example 3

### Polysorbate-80 AND Hydroxypropyl-ÿ-Cyclodextrin Enhance the Solubility of Compositions Comprising a Lipopeptide and an Antigen at 37° C. and Through Freeze/Thaw Cycling

To determine the effect of PS-80 and HPBCD on the solubility of Pam3Cys.M2e at 37° C. and through freeze/thaw cycling four test formulations were prepared as described below.

The test formulations were prepared at 1 mg/mL by dispensing Pam3Cys.M2e into room temperature formulation buffer and vortexing to mix. The formulations were then incubated overnight at 37° C. to examine the effect of increased temperature on solubility. The formulations were then filtered through a 33 mm diameter syringe filter with a 0.22 ÿm pore size PVDF membrane. After filtration each formulation was subjected to four freeze/thaw (F/T) cycles from room temperature to −2° C. The A280/A350 ratio was determined on the initial preparation, after 37° C. incubation, after filtration and after four freeze/thaw cycles. F107 was included in the study as a second control formulation that is based on a different buffer system (Tris/Histidine), a lower NaCl concentration (75 mM) and the use of 5% sucrose as a cryoprotectant.

The results (Table 3 below) indicate that the initial A280/A350 values were identical for F101 and F107, but significantly higher for F108 and F109. The A280/A350 ratio increased in each formulation when incubated overnight at 37° C. However, the A280/A350 ratios after 37° C. incubation were again significantly higher for F108 and F109 than for F101 and F107. After filtration the A280/A350 ratio increased for each formulation but the ratio remained significantly higher in F108 and F109 than in F101 or F107. After four freeze/thaw cycles the A280/A350 ratio decreased in F101 and F109 but increased or remained stable in F107 and F108. After 37° C. incubation, filtration and freeze/thaw cycling the highest A280/A350 values were recorded for the F108 and F109 formulations (>100 and 33, respectively), while PBS had the lowest A280/A350 ratio (10.9). These results indicate that PS-80 and HPBCD enhance the solubility of Pam3Cys.M2e at room temperature, at 37° C. and through freeze/thaw cycling.

TABLE 3
Effect of PS-80 and HPBCD on Pam3Cys.M2e Solubility.

| | A280/A350 ratio | | | |
|---|---|---|---|---|
| Formulation | initial | after 37° C. | after filtration | after F/T cycling |
| F101 | 4.8 | 6.5 | 19.8 | 10.9 |
| F107 | 4.8 | 6.5 | 15.8 | 16.5 |
| F108 | 8.8 | 17.3 | >100 | >100 |
| F109 | 6.8 | 11.2 | 67.3 | 33 |

### Example 4

### Polysorbate-80 and Hydroxypropyl-ÿ-Cyclodextrin Decrease the Particle Size of a Composition Comprising a Lipopeptide and an Antigen

A study was conducted for the purpose of evaluating the solubility, particle size and recovery through sterile filtration of four Pam3Cys.M2e test formulations (F106, F105, F110, and F111, see Table 1, supra). Each formulation was prepared by dissolving dry powder Pam3Cys.M2e directly in room temperature formulation buffer to a final peptide concentration of 1 mg/mL. Each sample was vigorously mixed by high speed vortexing for 1.5 min immediately after adding formulation buffer. The test formulations were then incubated at room temperature for approximately 2 hours prior to sterile filtration through a 33 mm diameter syringe filter with a 0.22 ÿm pore size PVDF membrane. UV spectra were taken before and after filtration to determine the A280/A350 ratio and the filtration recovery. Dynamic light scattering measurements were performed after filtration.

The results of the A280/A350 measurements and filtration recoveries (Table 4 below) indicate that the addition of PS-80 and/or HPBCD to the F106 formulation enhanced the solubility and filtration recovery of Pam3Cys.M2e. The highest A280/A350 ratio was obtained with the F111 formulation before (12.7) and after filtration (18.3). F111 was the only formulation in this study containing both PS-80 and HPBCD. Because the solubility of Pam3Cys.M2e varies from lot to lot, a second lot of Pam3Cys.M2e was formulated in F106 and F111 to determine the effectiveness of PS-80 and cyclodextrin for enhancing the solubility. The results (Table 4) indicate that although the initial A280/A350 values were nearly the same (4.4 vs. 3.7 for F106 and F111, respectively) the A280/A350 value for F111 was significantly higher than for F106 after filtration. Moreover, the filtration recovery of Pam3Cys.M2e in F111 was 92% vs. only 79% in F106. These results clearly show that PS-80 and cyclodextrin enhance the solubility of Pam3Cys.M2e.

TABLE 4
Effect of PS-80 and HPBCD on Pam3Cys.M2e Solubility.

| | A280/A350 ratio | | Filtration | Z-average |
|---|---|---|---|---|
| Formulation | Initial | after filtration | Recovery | Particle Size (nm) |
| F106 | 5.2 | 9.6 | 86% | 92 |
| F105 | 6.6 | 12.6 | 89% | 77 |
| F110 | 7.7 | 13.8 | 90% | 80 |
| F111 | 12.7 | 18.3 | 95% | 60 |
| F106* | 4.4 | 9.5 | 79% | 93 |
| F111* | 3.7 | 13.4 | 92% | 63 |

*Prepared with a different lot of Pam3Cys.M2e.

To determine the effect of PS-80 and HPBCD on the particle size of Pam3Cys.M2e dynamic light scattering (DLS) measurements were performed on the test formulations after the 0.22 ÿm filtration. The results (Table 4) indicate that after 0.22 ÿm filtration the particle size of Pam3Cys.M2e was largest in formulation F106 (92 and 93 nm Z-average) and smallest in formulation F111 (60 and 63 nm Z-average). These results show that PS-80 and HPBCD improve the solubility of Pam3Cys.M2e, increases recovery through 0.22 ÿm filtration and reduces the particle size.

### Example 5

### Polysorbate-80 and Hydroxypropyl-ÿ-Cyclodextrin Enhance the Solubility of Compositions Comprising a Lipopeptide and an Antigen

To determine the ability of PS-80 and cyclodextrin to enhance the solubility of lipopeptides other than Pam3Cys.M2e three different compositions comprising a lipopeptide and an antigen were formulated at 1 mg/mL in a formulation containing PS-80 and HPBCD (F111) and the same formulation without PS-80 and HPBCD (F106). First, a stock solution of each lipopeptide was prepared at 20 mg/mL in dimethylsulfoxide (DMSO). Then, each lipopeptide in DMSO was diluted to a final concentration of 1 mg/mL into F106 and F111. The lipopeptide solutions were then analyzed by dynamic light scattering to determine the Z-average particle size. The amino acid sequences of the three new lipopeptides are shown compared to Pam3Cys.M2e below. The results (Table 5) indicate that for each of the three lipopeptide sequences the Z-average particle size was significantly smaller in F111 than in F106.

Amino Acid Sequence of Lipopeptides

| | |
|---|---|
| Lipopeptide | Amino Acid Sequence |

| | | |
|---|---|---|
| Pam3Cys.M2e | SLLTEVETPIRNEWGSRSNDSSDP | (SEQ ID |
| | | NO: 1) |
| Pam3Cys.WNE.1 | ELEPPFGDSYIVVGRGEQQINHHWHKS | (SEQ ID |
| | | NO: 2) |
| Pam3Cys.JEE.1 | EMEPPFGDSYIVVGMGDKQINHHWHKA | (SEQ ID |
| | | NO: 3) |
| Pam3Cys.DEN.1 | EAEPPFGDSYIIIGVEPQQLKLNWFKK | (SEQ ID |
| | | NO: 4) |

TABLE 5
Effect of PS-80 and HPBCD on Lipopeptide Solubility.

| | |
|---|---|
| A280/A350 ratio | Z-Average (nm) |

| Lipopeptide | F106 | F111 | F106 | F111 |
|---|---|---|---|---|
| Pam3Cys.WNE.1 | 6.8 | 6.4 | 109 | 6 |
| Pam3Cys.JEE.1 | 27.1 | 31.8 | 374 | 17 |
| Pam3Cys.DEN.1 | 11.8 | 14.7 | 395 | 211 |

The UV absorbance of each lipopeptide solution was also determined and the A280/A350 ratios are shown in Table 5 above. The results indicate that for the Pam3Cys.JEE.1 and Pam3Cys.DEN.1 lipopeptides the presence of PS-80 and cyclodextrin in formulation F111 increased the A280/A350 ratio. For the Pam3Cys.WNE.1 lipopeptide the A280/A350 ratios in F106 and F111 are nearly the same, but the particle size was greatly reduced in the F111 formulation.

Taken together these results indicate that PS-80 and cyclodextrin significantly enhance the solubility and reduce the particle size of compositions comprising lipopeptides and an antigen in aqueous solution.

### Example 6

### Docusate Sodium and Dilauroylphosphoglycerol Enhance the Solubility of Compositions Comprising a Lipopeptide and an Antigen

To examine the effect of negatively charged surfactants on the solubility and particle size of compositions comprising a lipopeptide and an antigen, docusate sodium (DS) and dilauroylphosphoglycerol (1,2-Dilauroyl-sn-Glycero-3-[Phospho-rac-(1-glycerol)] (DLPG) were formulated with Pam3Cys.M2e in two different base formulations (F111 and F113). Each formulation was prepared by dissolving dry powder Pam3Cys.M2e directly in formulation buffer pre-warmed to 37° C. to a final peptide concentration of 1 mg/mL. Each sample was vigorously mixed by high speed vortexing immediately after adding formulation buffer. The test formulations were then incubated at 37° C. for approximately 1-3 hours prior to filtration through a 33 mm diameter syringe filter with a 0.22 ÿm pore size PVDF membrane. UV spectra were taken after filtration to determine the peptide concentration and the A280/A350 ratio. Dynamic light scattering measurements were performed after filtration to determine the Z-average particle size and the hydrodynamic diameter (Dh) of the major species by volume. The results (Table 6) indicate that the addition of DS to the F111 formulation significantly increased the A280/A350 ratio and decreased the Z-average particle size.

When Pam3Cys.M2e was formulated in F113, a formulation similar to F111 but containing no NaCl and 10% sucrose, the particle size and A280/A350 results were similar to the values obtained for Pam3Cys.M2e formulated in F111. Therefore, lowering the salt concentration and increasing the sucrose concentration did not significantly affect the particle size of Pam3Cys.M2e. However, when DS and DLPG were added to the F113 formulation a large increase in A280/A350 was observed. Moreover, the major particle size by volume decreased from 27 nm to 8-10 nm.

These results clearly show that the addition of DS and DLPG to the F111 and F113 formulations reduce the particle size and enhance the solubility of Pam3Cys.M2e.

TABLE 6
Effect of DS and DLPG on the Solubility of Pam3Cys.M2e

| | *Particle Size (Dh) | | UV absorbance |
|---|---|---|---|
| Formulation | Z-average (nm) | by volume (nm) | A280/A350 |
| F111 | 68 | 24 | 13.8 |
| F117 | 42 | 19 | 26.7 |
| F119 | 48 | 20 | 27.3 |
| F113 | 62 | 27 | 15.0 |
| F120 | 60 | 8 | 40.1 |
| F123 | 44 | 10 | 34.0 |

*Particle size was determined by dynamic light scattering. The hydrodynamic diameter of the particles is expressed in nm. Both the Z-average particle size and the size of the major species by volume are recorded.

### Example 7

### Docusate Sodium Enhances the Antigenicity of Compositions Comprising a Lipopeptide and an Antigen

The hydrophobic nature of the lipopeptide, Pam3Cys, of fusion protein Pam3Cys.M2e suggests that Pam3Cys.M2e peptides are likely to aggregate through hydrophobic interactions in aqueous solutions. The results described above, show that a composition comprising a lipopeptide (Pam3Cys) and an antigen (M2e), in particular, Pam3Cys.M2e, does aggregate to form particles in all of the formulations tested. Because aggregation of Pam3Cys.M2e peptides into a particle is likely to result in some of the M2e amino acid sequences being buried “inside” the particle rather than on the “surface” the antigenicity of Pam3Cys.M2e particles might be enhanced if more of the M2e sequences were available for antibody binding on the “surface” of the particle. Since DS enhances the solubility and reduces the particle size of Pam3Cys.M2e in F111 and F113 the ability of DS to enhance the availability of antigenic sequences of Pam3Cys.M2e was examined using the capture M2e ELISA.

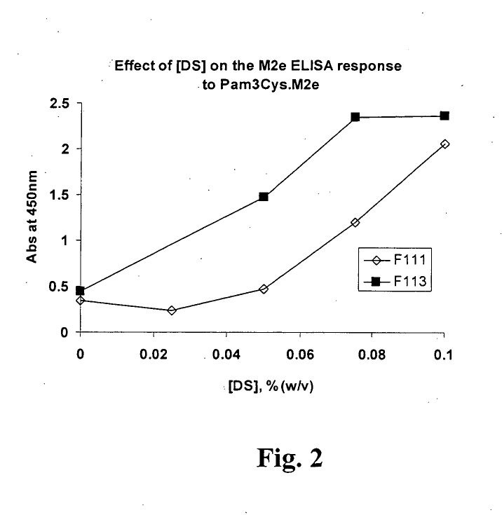

Pam3Cys.M2e was formulated at 1 mg/mL in either the F111 or F113 formulation containing increasing concentrations of DS. All peptide samples were diluted with 30% assay diluent (AD) to a peptide concentration of 1 ÿg/mL prior to applying the sample to the ELISA plate. Samples were probed with the monoclonal antibody 14C2b. The ELISA signal (Abs 450 nm) is plotted vs. the concentration of DS in FIG. 2. The results clearly show that the ELISA signal increases with increasing DS concentration in both F111 and F113 but the signal is higher at each DS concentration in F113. Therefore, the addition of DS to the F111 and F113 formulations results in more exposure of the 14C2 epitope on the surface of the Pam3Cys.M2e particles and indicates that DS enhances the antigenicity of Pam3Cys.M2e in vitro.

### Example 8

### Docusate Sodium Enhances the In Vivo Efficacy of a Composition Comprising a Lipopeptide and an Antigen

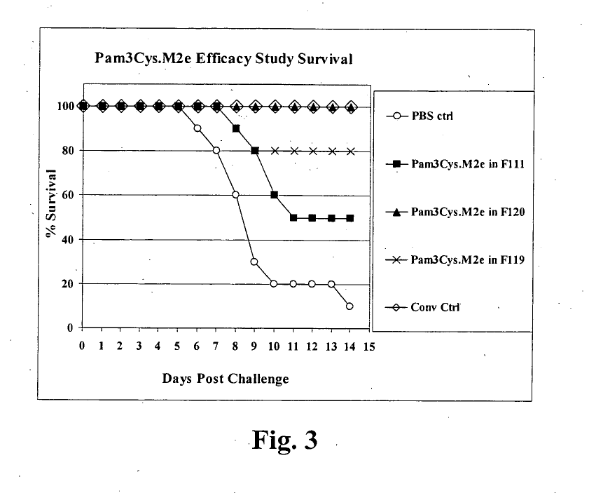

The efficacy of Pam3Cys.M2e (fusion protein of a lipopeptide and an antigen) as an influenza vaccine was evaluated in BALB/c mice using the well characterized mouse adapted strain, Influenza A/Puerto Rico/8/34 (PR/8) as the challenge virus. Groups of ten mice were immunized s.c. on day 0 and 14 with 30 ÿg of Pam3Cys.M2e in formulation F111 ( ), 30 ÿg of Pam3Cys.M2e in formulation F120 ( ), 30 ÿg of Pam3Cys.M2e in formulation F119 (X). A group receiving PBS alone was included as a negative control ( ), and a convalescent group with immunity to PR/8 following a sublethal challenge with the virus was included as a positive control ( ). On day 28, animals were challenge with an LD90 of the PR/8 challenge stock. Survival was followed for 14 days post challenge (FIG. 3).

As expected, animals in the convalescent group which had successfully cleared an earlier non-lethal infection with PR/8 demonstrated 100% protection to a subsequent viral challenge. Animals receiving the PBS buffer alone exhibited morbidity beginning on days 7 and 8, with 80% lethality occurring by day 10, while animals immunized with 30 ÿg of Pam3Cys.M2e in F111 demonstrated enhanced survival, with 50% of mice surviving the challenge. Animals receiving Pam3Cys.M2e in F119 exhibited morbidity beginning on days 8 and 9 with 80% of the mice surviving. Animals receiving Pam3Cys.M2e in F120 exhibited the mildest disease course with 90 to 100% of the mice in these groups surviving the lethal challenge. These results demonstrate that Pam3Cys.M2e can confer protective immunity to a challenge with influenza A in vivo and that the efficacy of Pam3Cys.M2e is enhanced by docusate sodium.

### EQUIVALENTS

While this invention has been particularly shown and described with references to preferred embodiments thereof, it will be understood by those skilled in the art that various changes in form and details may be made therein without departing from the scope of the invention encompassed by the appended claims.

What is claimed is:

1. A composition comprising a lipopeptide, an antigen and an emulsifying agent, wherein the lipopeptide includes at least one member selected from the group consisting of Pam3Cys and Pam2Cys.

2. The composition of claim 1, wherein the emulsifying agent includes at least one member selected from the group consisting of a polyoxyethylene sorbitan monoleate, a cyclodextrin, a docusate salt and a surfactant.

3. The composition of claim 1, wherein the lipopeptide and the antigen are components of a fusion protein.

4. The composition of claim 2, wherein the cyclodextrin includes a hydroxypropyl-β-cyclodextrin.

5. The composition of claim 2, wherein the docusate salt includes a sodium docusate.

6. The composition of claim 2, wherein the surfactant includes a negatively charged surfactant.

7. The composition of claim 6, wherein the negatively charged surfactant includes a Dilauroylphosphoglycerol (1 ,2-Dilauroyl-sn-Glycero-3-[Phospho-rac-( 1-glycerol)].

8. A composition comprising a lipopeptide, an antigen, a polyoxyethylene sorbitan monoleate, a cyclodextrin, a docusate salt and a surfactant.

9. The composition of claim 8, wherein the lipopeptide includes at least one member selected from the group consisting of Pam3Cys and Pam2Cys.

10. A composition comprising a lipopeptide, an antigen and a polyoxyethylene sorbitan monoleate, wherein the lipopeptide includes at least one member selected from the group consisting of Pam3Cys and Pam2Cys.

11. A composition comprising a lipopeptide, an antigen and a docusate salt, wherein the lipopeptide includes at least one member selected from the group consisting of Pam3Cys and Pam2Cys.

12. A composition comprising a lipopeptide, an antigen and a surfactant, wherein the lipopeptide includes at least one member selected from the group consisting of Pam3Cys and Pam2Cys.

13. A composition comprising a lipopeptide, an antigen and a cyclodextrin, wherein the lipopeptide includes at least one member selected from the group consisting of Pam3Cys and Pam2Cys.

14. A method of stimulating an immune response in a subject, comprising the step of administering to the subject a composition that includes a lipopeptide, an antigen and an emulsifying agent, wherein the lipopeptide includes at least one member selected from the group consisting of Pam3Cys and Pam2Cys.

15. The method of claim 14, wherein the emulsifying agent includes at least one member selected from the group consisting of a polyoxyethylene sorbitan monoleate, a cyclodextrin, a docusate salt and a surfactant.

16. The method of claim 14, wherein the lipopeptide and the antigen are components of a fusion protein.

17. The method of claim 15, wherein the cyclodextrin includes a hydroxypropyl-β-cyclodextrin.

18. The method of claim 15, wherein the docusate salt includes sodium docusate.

19. The method of claim 15, wherein the surfactant includes a negatively charged surfactant.

20. The method of claim 19, wherein the negatively charged surfactant includes Dilauroylphosphoglycerol (1,2-Dilauroyl-sn-Glycero-3-[Phospho-rac-(1-glycerol)].

21. A method of stimulating an immune response in a subject, comprising the step of administering to the subject a composition that includes a lipopeptide, an antigen, a polyoxyethylene sorbitan monoleate, a cylcodextrin, a docusate salt and a surfactant.

22. The method of claim 19, wherein the lipopeptide includes at least one member selected from the group consisting of Pam3Cys and Pam2Cys.

23. A method of stimulating an immune response in a subject, comprising the step of administering to the subject a composition that includes a lipopeptide, an antigen and a polyoxyethylene sorbitan monoleate, wherein the lipopeptide includes at least one member selected from the group consisting of Pam3Cys and Pam2Cys.

24. A method of stimulating an immune response in a subject, comprising the step of administering to the subject a composition that includes a lipopeptide, an antigen and a docusate salt, wherein the lipopeptide includes at least one member selected from the group consisting of Pam3Cys and Pam2Cys.

25. A method of stimulating an immune response in a subject, comprising the step of administering to the subject a composition that includes a lipopeptide, an antigen and a surfactant, wherein the lipopeptide includes at least one member selected from the group consisting of Pam3Cys and Pam2Cys.

26. A method of stimulating an immune response in a subject, comprising the step of administering to the subject a composition that includes a lipopeptide, an antigen and a cyclodextrin, wherein the lipopeptide includes at least one member selected from the group consisting of Pam3Cys and Pam2Cys.
